(19) United States
(12) Patent Application Publication
(10) Pub. No.: US 2006/0134047 A1
(43) Pub. Date: Jun. 22, 2006

(54) Personal care and household compositions of hydrophobically-modified polysaccharides

(76) Inventors: Kirill N. Bakeev, Newark, DE (US); Paquita Erazo-Majewicz, Newark, DE (US); Jashawant J. Modi, Hockessin, DE (US); Tuyen T. Nguyen, Newark, DE (US); Hassan H. Rmaile, Wilmington, DE (US)

Correspondence Address: Hercules Incorporated;Hercules Plaz, 1313 N. Market Street, Wilmington, DE, 19894-0001, US

(21) Appl. No.: 11/300,628

(22) Filed: Dec. 14, 2005

Related U.S. Application Data
(60) Provisional application No. 60/636,682, filed on Dec. 16, 2004.

(51) Int. Cl. A61K 8/73

(52) U.S. Cl. 424/70.13

(57) ABSTRACT

A conditioning composition is used in functional systems (personal care and household care compositions) that has a nonionic hydrophobically modified cellulose ether (HMCE) having a weight average molecular weight (Mw) with a lower limit of 400,000 and an upper limit of 2,000,000 and a hydrophobic substitution lower limit of 0.6 wt % and an upper limit amount which renders said cellulose ether insoluble in a 5 wt % solution of surfactant and less than 0.05% by weight soluble in water and wherein the cellulose ether provides conditioning benefit to the functional system substrate. This composition has at least one active functional system ingredient.

This application claims the benefit of U.S. Provisional Application No. 60/636,682 filed Dec. 16, 2004.

### FIELD OF THE INVENTION

The present invention is related to the use of nonionic hydrophobically modified polysaccharides in personal care and household care compositions; and more specifically, it relates to the use in such compositions of hydrophobically-modified cellulose ethers, such as hydrophobically-modified hydroxyethylcellulose (HMHEC) polymers that show pronounced syneresis in aqueous solutions or in the presence of surfactants, including nonionic surfactants and anionic surfactants such as lauryl sulfate (LS) and lauryl ether sulfate (LES) surfactants.

### BACKGROUND

In the prior art, the commonly used approach to deliver a polymer coating from personal care or household compositions is through the use of complex formation between a cationic polymer and an anionic surfactant. It is well-known that the mechanism of conditioning for polymers with cationic functionality in hair care, cleansing skin care, and fabric care applications is based on dilution deposition of a cationic polymer-anionic surfactant complex that has both cationic polymer and oppositely charged surfactant. (U.S. Pat. No. 5,422,280) As the result of this mechanism, commercial products such as cationic guars, cationic hydroxyethylcellulose, and synthetic cationic polymers show high efficacy in conditioning shampoos, skin care cleansing formulations, and fabric cleansing/conditioning formulations.

In personal care applications, such as in hair care and skin care, and in household care applications, such as fabric care applications, there is a desire to deposit a coating onto the substrate, that reduces the energy needed to move a comb through hair in the wet or dry state or delivers a silky, soft feel to skin or to fabric. This coating can also act to improve the luster and moisture retention of hair and skin, as well as their manageability and feel.

The discovery of the improved deposition of silicone resins from cleansing formulations such as shampoos, using cationic polymer-anionic surfactant complexes has lead to the development of this approach to deliver hair conditioning, skin, and fabric conditioning. However, the tendency for silicone buildup on the hair after repeated washing with silicone shampoos, and the desire for clear conditioning formulations has left a strong market need for alternative approaches to achieve silicone-like conditioning on hair, skin, and fabric substrates with or without silicone resins, and without cationic polymers.

Hence, there is also a need in personal care applications for improved overall conditioning performance combined with other desirable attributes such as improved hair volume, manageability, hair repair, or color retention, skin moisturization and moisture retention, fragrance retention, sunscreen longevity on hair, skin, and fabrics, flavor enhancement and antimicrobial performance in oral care applications, and in household applications there is a need for fabric abrasion resistance and colorfastness.

Prior to the present invention, water soluble polysaccharides have been used in personal care applications, such as cleansing and cosmetic skincare, hair care, and oral care applications and in household applications such as cleaning, sanitizing, polishing, toilet preparations, and pesticide preparations; applications such as air deodorants/fresheners, rug and upholstery shampoos, insect repellent lotions, all purpose kitchen cleaner and disinfectants, toilet bowl cleaners, fabric softener-detergent combinations, fabric softeners, fabric sizing agents, dishwashing detergents, vehicle cleaners and shampoos. Widely used commercially available polysaccharides include water soluble polysaccharide ethers such as methyl cellulose (MC), hydroxypropylmethylcellulose (HPMC), hydroxyethylcellulose (HEC), hydroxypropylcellulose (HPC), ethylhydroxyethylcellulose (EHEC), hydroxypropyl (HP) guar, hydroxyethyl guar, guar, starch, and other nonionic starch and guar derivatives.

U.S. Pat. Nos. 5,106,609, 5,104,646, 6,905,694, and 5,100,658 are examples of patents that disclose the use of hydrophobically modified cellulose ethers in cosmetic products. These patents disclose the use of high molecular weight (i.e., 300,000 to 700,000) and alkyl carbon substitution in the hydrophobe (i.e., 3 to 24 carbons) for use in cosmetic compositions. U.S. Pat. No. 4,243,802 discloses a hydrophobically modified nonionic, water-insoluble, surfactant-soluble cellulose ether composition. The use of this material to increase the viscosity of an acidic shampoo composition and to emulsify oil in water emulsions is mentioned. Also, U.S. Pat. Nos. 4,228,277 and 4,352,916 describe hydrophobically modified cellulose ether derivatives, modified with long chain alkyl group substitution in the hydrophobe. U.S. Pat. No. 5,512,091 discloses hydrogel compositions containing water-insoluble hydrophobically-modified cellulose ethers. Publication US2001/0043912 discloses anti-frizz hair care compositions containing a hydrophobically-modified cellulose ether thickener. U.S. Pat. No. 4,845,207 discloses a hydrophobically modified nonionic, water-soluble cellulose ether and U.S. Pat. No. 4,939,192 discloses the use of such ether in building compositions. U.S. Pat. No. 4,960,876 discloses hydrophobically-modified galactomannan compositions as thickeners for use in paint, paper, and ceramic applications. U.S. Pat. No. 4,870,167 discloses hydrophobically-modified nonionic polygalactomannan ethers prepared from long-chain aliphatic epoxides, and suggests their possible use in cosmetics, including hand lotions, shampoos, hair treatment compounds, toothpastes, and gels for cleaning teeth. U.S. Pat. No. 6,387,855 discloses aqueous compositions containing silicone, a surfactant, and a hydrophobic galactomannan gum for washing and conditioning keratin.

The performance of water-soluble and water-insoluble hydrophobically-modified celluloses has been found lacking in terms of their ability to confer significant and predictable conditioning to keratin substrates. Hence, a need still exists in the industry to have cellulose ethers that confer significant and predictable conditioning to keratin substrates, and deposit films onto solid substrates such as fabrics, when delivered from aqueous compositions.

### SUMMARY OF THE INVENTION

The present invention is directed to a conditioning composition comprising:

(a) an aqueous based functional system selected from the group consisting of personal care products and household care products and

(b) a nonionic hydrophobically modified cellulose ether (HMCE) having a weight average molecular weight (Mw) with a lower limit of 400,000 and an upper limit of 2,000,000 and a hydrophobic substitution lower limit of 0.6 wt % and an upper limit amount which renders said cellulose ether soluble in a 5 wt % solution of surfactant and less than 0.05% by weight soluble in water or in a 1 wt % surfactant solution and wherein the cellulose ether provides conditioning benefit to a functional system substrate, and

(c) at least one active functional system active ingredient.

The present invention is also directed to a process of conditioning an aqueous based functional system selected from the group consisting of personal care and household care products comprising adding and mixing a sufficient amount of a hydrophobically modified cellulose ether that is compatible with the aqueous based functional system to thicken the functional system wherein the hydrophobically modified cellulose ether is a nonionic hydrophobically modified cellulose ether (HMCE) having a weight average molecular weight (Mw) with a lower limit of 400,000 and an upper limit of 2,000,000 and a hydrophobic substitution lower limit of 0.6 wt % and an upper limit amount which renders said cellulose ether soluble in a 5 wt % solution of surfactant and less than 0.05% by weight soluble in water or in a 1 wt % surfactant solution and wherein the cellulose ether provides conditioning benefit to a functional system substrate, and the resulting functional system has comparable or better conditioning properties as compared to when using similar thickening agents outside the scope of the present composition.

a. The hydrophobically modified polysaccharide polymers of the present invention can be either water-soluble with the formation of a homogeneous gel above a certain (critical) polymer concentration in water or partially soluble in water, (reaching a solution) dissolving with the help of anionic surfactant. In both cases the critical requirement to this polymer is syneresis upon dilution below a certain critical polymer concentration. Such polymers are useful as conditioning agents in 2-in-1 shampoos, in body cleansing formulations, in oral care cleansing systems such as dentifrices, and in fabric cleansing-conditioning systems due to their unique mechanism of activity and dilution-deposition upon rinsing.

b. By syneresis and dilution-deposition is meant that the hydrophobically modified polysaccharide whose original concentration is between 0.05%-10% by weight, undergoes liquid-gel phase separation (syneresis) in aqueous solutions when diluted to a final concentration with a lower limit of 0.01% by weight in solution. The discussed polymers are water-soluble with the formation of a homogeneous gel above a certain (critical) concentration in water of 0.1%-1%. The critical and unique requirement of these gels is syneresis upon dilution below certain critical concentration in the personal care composition. These polymers can be synthesized by methods known in the prior art.

c. In addition to polymer, the aqueous solution can include surfactant/water mixtures, cyclodextrin/surfactant/water mixtures, water-miscible solvents, salts, water soluble nonionic, cationic, or anionic polymers, and a combination of any of these.

### DETAILED DESCRIPTION OF THE INVENTION

It has been found that if a hydrophobically-modified polysaccharide polymer undergoes syneresis upon dilution in aqueous solution, the hydrophobically-modified polysaccharide polymer can deposit with high efficacy on substrates such as hair, skin, teeth, oral mucosa, or textile fabrics and can impart great conditioning benefits to the substrates. Upon deposition onto the substrate, the hydrophobically modified polysaccharide can also deposit other ingredients, which improve the condition or enhance the characteristics of the substrate. These polymers also have potential for conditioning skin from cleansing formulations or moisturizing formulations, since these polymers may also better deliver the oil phase typically used in creams and lotions.

Surprisingly, it has been found that nonionic hydrophobically modified polysaccharides, preferably cellulose derivatives and more specifically hydrophobically-modified hydroxyethylcellulose, HMHEC, that show pronounced syneresis in aqueous solution upon dilution can deposit with high efficacy on hair/skin and can impart great conditioning benefits to keratin substrates. Such polymers impart other benefits in hair styling, body lotions and sunscreens due to hydrophobic film formation on keratin substrates that would act as barrier between the surfaces and the surrounding atmosphere.

These polymers may also be useful as film-formers and co-deposition agents onto the surfaces of hair, skin, and textiles, aiding in protection of the hair, skin, and textile substrates from moisture-loss, aiding deposition of sunscreens and subsequent protection of these substrates from UV radiation, enhancing deposition of fragrance or flavor onto substrates and entrapping fragrance and flavor leading to their improved longevity on these substrates, or aiding deposition of antimicrobial reagents and other active personal care ingredients, resulting in improved longevity of the active on the substrate. In addition, these polymers find use in oral care applications such as dentifrices and denture adhesives to deliver prolonged flavor retention and flavor release. Prolonged release of antimicrobial and biocide agents from these polymers may also find usefulness in household and personal care applications, such as skin and hair treatment formulas and in oral care applications such as dentifrice, denture adhesives, and whitening strips.

In accordance with this invention, the conditioning benefits of hydrophobically modified polysaccharides, preferable hydrophobically-modified cellulose ether polymers, are demonstrated as conditioning agents in personal care compositions such as hair care, skin care, and oral care compositions as well as household care compositions, such as laundry cleaner and softener products for textile substrates and hard surface cleaner products.

In accordance with the present invention, the functional system substrate is defined as a material that is related to personal care and household care applications. In personal care, the substrate can be skin, hair, teeth, and mucous membranes. In household care products, the substrate can be hard surfaces such as metals, marbles, ceramics, granite, wood, hard plastics, and wall boards or textiles fabrics.

Any water soluble polysaccharide or derivatives can be used as the backbone to form the hydrophobically modified polysaccharide of this invention. Thus, e.g., hydroxyethylcellulose (HEC), hydroxypropylcellulose (HPC), methylcellulose (MC), hydroxypropylmethylcellulose (HPMC), ethylhydroxyethylcellulose (EHEC), and methylhydroxyethylcellulose (MHEC) and, agar, dextran, starch, and their nonionic derivatives can all be modified. The amount of nonionic substituent such as methyl, hydroxyethyl, or hydroxypropyl does not appear to be critical so long as there is a sufficient amount to assure that the ether is water soluble. The polysaccharides of this invention have a sufficient degree of nonionic substitution to cause them to be water soluble and a hydrophobic moiety including 1) 3-alkoxy-2-hydroxypropyl group wherein the alkyl moiety is a straight or branched chain having 3-30 carbon atoms, or 2) C₃-C₃₀ alkyl, and C₇-C₃₀ aryl, aryl alkyl, and alkyl aryl groups and mixtures thereof, wherein the hydrophobic moiety is present in an amount up to the amount that produces a hydrophobically-modified polysaccharide that shows pronounced syneresis in aqueous solution or in the presence of anionic surfactants such as, for example, lauryl sulfate (LS) and lauryl ether sulfate (LES) surfactants. When the hydrophobe is an alkyl moiety, the number of carbons can be 3-30, preferably 6-22, more preferably 8-18, and most preferably 10-16. The aryl, aryl alkyl, or alkyl aryl moiety can have an upper limit carbon amount of 30 carbons, preferably 22 carbons, more preferably 18 carbons, and even more preferably 16 carbons. The lower limit of the carbon amount is 7 carbons, more preferably 8 carbons, and even more preferably 10 carbons.

The preferred polysaccharide backbone is hydroxyethylcellulose (HEC). The HEC which is modified to function in this invention is a commercially available material. Suitable commercially available materials are marketed by the Aqualon Company, a division of Hercules Incorporated, Wilmington, Del. U.S.A., under the trademark Natrosol®.

The alkyl modifier can be attached to the polysaccharide backbone via an ether, ester, or urethane linkage. Ether is the preferred linkage as the reagents most commonly used to effect etherification because it is readily obtainable; the reaction is similar to that commonly used for the initial etherification, and the reagents used in the reaction are usually more easily handled than the reagents used for modification via the other linkages. The resulting linkage is also usually more resistant to further reactions.

An example of the polysaccharide of the present invention is the 3-alkoxy-2-hydroxypropylhydroxyethylcellulose that shows pronounced syneresis in aqueous solution or in the presence of nonionic surfactants, such as acetylene based surfactants, or in the presence of anionic surfactants such as, for example, lauryl sulfate (LS) and lauryl ether sulfate (LES) surfactants.

The hydrophobic moiety is generally contained in an amount of from about 0.6 wt % to an upper limit amount which renders said hydrophobically modified polysaccharide soluble in a 5 wt % solution of surfactant, and less than 0.05 wt % soluble in water or in a 1 wt % surfactant solution. The alkyl group of the 3-alkoxy-2-hydroxypropyl group can be a straight or branched chain alkyl group having 3 to 30 carbon atoms. Exemplary modifying radicals are propyl-, butyl-, pentyl-, 2-ethylhexyl, octyl, cetyl, octadecyl, methylphenyl, and docosapolyenoic glycidyl ether.

The hydrophobically modified polysaccharide of the present invention is an essential ingredient of the system. An optional ingredient that may be in the system is a surfactant that can be either soluble or insoluble in the composition. Another optional ingredient is a compatible solvent may also be used in the system that can be either a single solvent or a blend of solvents.

Examples of the surfactants are anionic, nonionic, cationic, zwitterionic, or amphoteric type of surfactants, and blends thereof. Except for cationic surfactants, the surfactant can be soluble or insoluble in the present invention and (when used) is present in the composition in the amount of from 0.01 to about 50 wt % by weight of the composition. Synthetic anionic surfactants include alkyl and alkyl ether sulfates. Cationic surfactants can be present in an amount of from 0.01 to about 1.0 wt %

Nonionic surfactants, can be broadly defined as compounds containing a hydrophobic moiety and a nonionic hydrophilic moiety. Examples of the hydrophobic moiety can be alkyl, alkyl aromatic, dialkyl siloxane, polyoxyalkylene, and fluoro-substituted alkyls. Examples of hydrophilic moieties are polyoxyalkylenes, phosphine oxides, sulfoxides, amine oxides, and amides. Nonionic surfactants such as those marketed under the trade name Surfynol® are also useful in this invention.

Cationic surfactants useful in vehicle systems of the compositions of the present invention, contain amino or quaternary ammonium hydrophilic moieties which are positively charged when dissolved in the aqueous composition of the present invention.

Zwitterionic surfactants are exemplified by those which can be broadly described as derivative of aliphatic quaternary ammonium, phosphonium, and sulfonium compounds, which can be broadly described as derivative of aliphatic quaternary ammonium, phosphonium, and sulfonium compounds, in which the aliphatic radicals can be straight or branched chain, and wherein one of the aliphatic substituents contains from about 8 to about 18 carbon atoms and one contains as anionic water-solubilizing group, e.g., carboxy, sulfonate, sulfate, phosphate, or phosphonate.

Examples of amphoteric surfactants which can be used in the vehicle systems of the compositions of the present invention are those which are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate.

According to the present invention, the solvent used in the system should be compatible with the other components of the present composition. Examples of the solvents that may be used in the present invention are water, water-lower alkanols mixtures, and polyhydric alcohols having from 3 to 6 carbon atoms and from 2 to 6 hydroxyl groups. Preferred solvents are water, propylene glycol, water-glycerine, sorbitol-water, and water-ethanol. The solvent (when used) in the present invention is present in the composition at a level of from 0.1% to 99% by weight of the composition.

In certain instances, the active component is optional because the dissolved polymer can be the active ingredient component. An example of this is the use of the polymer in a conditioner formulation for hair or skin conditioning or in a fabric conditioner formulation. However, when an active ingredient is needed, it must provide some benefit to the user or the user's body.

In accordance with the present invention, the functional system may be either a personal care product or a household care product. When the functional system is a personal care product that contains at least one active personal care ingredient, the personal care active ingredient includes, but is not limited to, analgesics, anesthetics, antibiotic agents, antifungal agents, antiseptic agents, antidandruff agents, antibacterial agents, vitamins, hormones, antidiarrhea agents, corticosteroids, anti-inflammatory agents, vasodilators, kerolytic agents, dry-eye compositions, wound-healing agents, anti-infection agents, as well as solvents, diluents, adjuvants and other ingredients such as water, ethyl alcohol, isopropyl alcohol, propylene glycol, higher alcohols, glycerine, sorbitol, mineral oil, preservatives, surfactants, propellants, fragrances, essential oils, and viscosifying agents.

Personal care compositions include hair care, skin care, sun care, nail care, and oral care compositions. Examples of active substances that may suitably be included, but not limited to, in the personal care products according to the present invention are as follows:

1) Perfumes, which give rise to an olfactory response in the form of a fragrance and deodorant perfumes which in addition to providing a fragrance response can also reduce body malodor;

2) Skin coolants, such as menthol, menthyl acetate, menthyl pyrrolidone carboxylate N-ethyl-p-menthane-3-carboxamide and other derivatives of menthol, which give rise to a tactile response in the form of a cooling sensation on the skin;

3) Emollients, such as isopropylmyristate, silicone materials, mineral oils and vegetable oils which give rise to a tactile response in the form of an increase in skin lubricity;

4) Deodorants other than perfumes, whose function is to reduce the level of or eliminate micro flora at the skin surface, especially those responsible for the development of body malodor. Precursors of deodorants other than perfume can also be used;

5) Antiperspirant actives, whose function is to reduce or eliminate the appearance of perspiration at the skin surface;

6) Moisturizing agents, that keep the skin moist by either adding moisture or preventing from evaporating from the skin;

7) Cleansing agents, that remove dirt and oil from the skin;

8) Sunscreen active ingredients that protect the skin and hair from UV and other harmful light rays from the sun. In accordance with this invention a therapeutically effective amount will normally be from 0.01 to 10% by weight, preferable 0.1 to 5% by weight of the composition;

9) Hair treatment agents, that condition the hair, cleanse the hair, detangles hair, acts as styling agent, volumizing and gloss agents, color retention agent, anti-dandruff agent, hair growth promoters, hair dyes and pigments, hair perfumes, hair relaxer, hair bleaching agent, hair moisturizer, hair oil treatment agent, and antifrizzing agent;

10) Oral care agents, such as dentifrices and mouth washes, that clean, whiten, deodorize and protect the teeth and gum;

11) Denture adhesives that provide adhesion properties to dentures;

12) Shaving products, such as creams, gels and lotions and razor blade lubricating strips;

13) Tissue paper products, such as moisturizing or cleansing tissues;

14) Beauty aids, such as foundation powders, lipsticks, and eye care; and

15) Textile products, such as moisturizing or cleansing wipes.

In accordance with the present invention, when the functional system is a household care compositions, this household care product includes a hydrophobically modified polysaccharide and at least one active household care ingredient. The household care active ingredient must provide some benefit to the user. Examples of active substances that may suitably be included, but not limited to, according to the present invention are as follows:

1) Perfumes, which give rise to an olfactory response in the form of a fragrance and deodorant perfumes which in addition to providing a fragrance response can also reduce odor;

2) Insect repellent agent whose function is to keep insects from a particular area or attacking skin;

3) Bubble generating agent, such as surfactant that generates foam or lather;

4) Pet deodorizer or insecticides such as pyrethrins that reduces pet odor;

5) Pet shampoo agents and actives, whose function is to remove dirt, foreign material and germs from the skin and hair surfaces;

6) Industrial grade bar, shower gel, and liquid soap actives that remove germs, dirt, grease and oil from skin, sanitizes skin, and conditions the skin;

7) All purpose cleaning agents, that remove dirt, oil, grease, and germs from the surface in areas such as kitchens, bathroom, and public facilities;

8) Disinfecting ingredients that kill or prevent growth of germs in a house or public facility;

9) Rug and Upholstery cleaning actives which lift and remove dirt and foreign particles from the surfaces and also deliver softening and perfumes;

10) A laundry softener active, which reduces static and makes fabric feel softer;

11) Laundry detergent ingredients which remove dirt, oil, grease, stains and kills germs;

12) Laundry or detergent or fabric softener ingredients that reduce color loss during the wash, rinse, and drying cycle of fabric care;

13) Dishwashing detergents which remove stains, food, germs;

14) Toilet bowl cleaning agents, which remove stains, kills germs, and deodorizes;

15) Laundry prespotter actives which helps in removing stains from clothes;

16) Fabric sizing agent which enhances appearance of the fabric;

17) Vehicle cleaning actives which removes dirt, grease, etc. from vehicles and equipment;

18) Lubricating agent which reduces friction between parts; and

19) Textile products, such as dusting or disinfecting wipes.

The above lists of personal care and household care active ingredients are only examples and are not complete lists of active ingredients that can be used. Other ingredients that are used in these types of products are well known in the industry. In addition to the above ingredients conventionally used, the composition according to the present invention can optionally also include ingredients such as a colorant, preservative, antioxidant, nutritional supplements, alpha or beta hydroxy acid, activity enhancer, emulsifiers, functional polymers, viscosifying agents (such as salts, i.e., NaCl, NH₄Cl, and KCl, water-soluble polymers, i.e., hydroxyethylcellulose and hydroxypropylmethylcellulose, and fatty alcohols, i.e., cetyl alcohol), alcohols having 1-6 carbons, fats or fatty compounds, antimicrobial compound, zinc pyrithione, silicone material, hydrocarbon polymer, emollients, oils, surfactants, medicaments, flavors, fragrances, suspending agents, and mixtures thereof.

In accordance with the present invention, examples of functional polymers that can be used in blends with the hydrophobically modified polysaccharides or derivatives thereof of this invention include water-soluble polymers such as acrylic acid homopolymers such as Carbopol® product and anionic and amphoteric acrylic acid copolymers, vinylpyrrolidone homopolymers and cationic vinylpyrrolidone copolymers; nonionic, cationic, anionic, and amphoteric cellulosic polymers such as hydroxyethylcellulose, hydroxypropylcellulose, carboxymethylcellulose, hydroxypropylmethylcellulose, cationic hydroxyethylcellulose, cationic carboxymethylhydroxyethylcellulose, and cationic hydroxypropylcellulose; acrylamide homopolymers and cationic, amphoteric, and hydrophobic acrylamide copolymers, polyethylene glycol polymers and copolymers, hydrophobic polyethers, hydrophobic polyetheracetals, hydrophobically-modified polyetherurethanes and other polymers referred to as associative polymers, hydrophobic cellulosic polymers, polyethyleneoxide-propylene oxide copolymers, and nonionic, anionic, hydrophobic, amphoteric, and cationic polysaccharides such as xanthan, chitosan, carboxymethyl guar, alginates, gum arabic, hydroxypropyl guar, hydrophobic guar polymers, carboxymethyl guar hydroxypropyltrimethylammonium chloride, guar hydroxypropyltrimethylammonium chloride, and hydroxypropyl guar hydroxypropyltrimethylammonium chloride.

In accordance with the invention, the silicone materials which can be used are polyorganosiloxanes that can be in the form of polymers, oligomers, oils, waxes, resins, or gums or polyorganosiloxane polyether copolyols, amodimethicohies, cationic polydimethylsiloxane materials and any other silicone material that is used in personal care or household compositions.

The polymers of the present invention are water-soluble with the formation of a homogeneous gel above a certain (critical) concentration in water of 0.01%-1%. The critical and unique requirement of these gels is syneresis upon dilution below certain critical concentration in the personal care composition. These polymers can be synthesized by methods known in the prior art.

Other water-insoluble HMHECS that formed gels or solutions in surfactant/water or ethanol/water mixtures, and syneresis upon dilution below certain critical concentration in the personal care composition, are also useful in this invention. The polymers of this invention can be useful as conditioning agents in 2-in-1 shampoos, body lotions, sunscreens, antifrizz and hair styling. The polymers of this invention can also be used to improve hair volume, manageability, hair repair, or color retention, skin moisturization and moisture retention, fragrance retention, sunscreen longevity on hair, skin, and fabrics, flavor enhancement and antimicrobial performance in oral care applications, and improve fabric abrasion resistance and colorfastness in household applications.

For a more detailed understanding of the invention, referenced can be made to the following examples which are intended as further illustrations of the invention but are not to be construed in a limiting sense. All parts and percentages are by weight unless stated otherwise.

### EXAMPLES

Wet and dry hair combability measurements are typical test methods used to measure conditioning performance in shampoo and conditioner applications. In skin care applications, skin lubricity or reduced friction or softer feel of the skin, reduced water vapor transmission and improved skin elasticity are test methods used to measure skin conditioning. In surfactant-based household cleansing product formulations where conditioning performance is desired, such as dish detergents, fabric softeners, and antistatic products, conditioning refers to imparting a softer feel to fabric and eliminating static effects, eliminating fabric fiber breakage or deformation known as pilling. Imparting color retention properties to fabrics is also important and can be measured.

### Standard Testing Procedures

Silicone deposition can be measured by several techniques. One technique used for quantifying silicone deposition for Examples of the invention is described as follows:

### Silicone Deposition Measurement

Each 2-5 gram sample was weighed to the nearest mg, after removal of sample holder, and placed into clean 8 oz jars with approximately 150 ml of methylene chloride. The samples were shaken for 1.5 hours at room temperature. The methylene chloride supernatant was filtered using Whatman # 41 filter paper and quantitatively transferred to clean 8 oz jars and evaporated to dryness with mild heat and a nitrogen sparge. Each sample was then dissolved into 2 ml of chloroform-d and quantitatively transferred to a 5-ml volumetric flask. Three chloroform-d rinses were used to transfer each sample to the 5-ml volumetric flask. All flasks were diluted to the mark with solvent and inverted. Each sample was examined in a NICOLET MAGNA 550 FT-IR with 150 co-added scans at 4 cm⁻¹ resolution and 0.4747 velocity using a 0.1 cm-fixed path salt cell. A chloroform-d reference spectrum was used to subtract out the solvent bands (diff=1.0). The silicone level was determined by measuring the peak height of the Si—CH₃ stretch at 1260 cm⁻¹ (baseline 1286 and 1227 cm⁻¹) followed by conversion to mg/ml of silicone using a low level calibration curve extending from 10-300 parts per million (ppm). Each sample was corrected for dilution volume and sample weight. All values are reported to the nearest ppm.

Formulation I
Surfactant Premix

| | Grams | % active | |
|---|---|---|---|
| ALS¹ | 654 | 11.44643 | Stepanol AM |
| ALES² | 213 | 3.727966 | Steol CA-330 |
| CAPB³ | 175 | 3.062883 | Amphosol CA |
| Coco MEA⁴ | 16 | | |
| DI Water | 543.6 | | |

Wt % Ingredient in shampoo⁵

| | |
|---|---|
| ALS | 8.699287 |
| ALES | 2.833254 |
| CAPB | 2.327791 |
| Total | 13.86033 |

¹Ammonium Lauryl Sulfate - Stepanol AM (Stepan)
²Ammonium Laureth Sulfate (3 EO) - Steol CA-330 (Stepan)
³Cocamidopropyl betaine - Amphosol CA (Stepan)
⁴Coco Monoethanolamide - Ninol CMP (Stepan)
⁵Use 76 grams premix per 100 grams shampoo

Procedure for Preparing Silicone Shampoos from Premix Formulation I—Lightly Bleached European Medium Brown Hair

76 grams of Formulation I surfactant premix were weighed into a 4-oz. glass jar. 10 grams of 2 wt % polymer solutions and 9 grams additional water where then weighed into the 4-oz. jar containing the 76 grams Formulation I surfactant premix. The 4-oz jar was then clamped into a 60° C. water bath. A twin-propeller mixer was lowered into the jar and the jar opening was covered with a lid to reduce evaporation loss.

The sample was stirred for 15-minutes. After the 15-minutes of stirring, 0.25 g of NH₄Cl (ammonium chloride Baker reagent) was added to the jar. The sample was then stirred for an additional 45 minutes while covered. The sample jar was then removed from the 60° C. bath. The jar was then clamped into a room temperature water bath. The overhead stirrer was reattached and the stirring of the sample was begun in the water bath. The sample was allowed to stir for a minimum of 5-minutes. This was sufficient time for the sample temperature to drop below 35° C.

3.68 g of dimethicanol GESM555 silicone was added to the jar and the jar was stirred for a minimum of 5-minutes additionally. 0.5 g of Germaben® II product was added to the jar and the jar was stirred for an additional minimum amount of time of 5-minutes.

The pH was checked and adjusted to 6.2-6.5 (either a 10% or 50% solution of citric acid was used to lower the pH). The jar was sealed and centrifuged for about 10-minutes at 3,000 rpm to remove any entrapped air.

The Brookfield viscosity equilibration was measured for 1 hour on a Brookfield LV-4, at 25.0° C., @ 0.3 RPM, then 12 RPM, then 30 RPM. A 3-minute rotation time was used at each speed.

Procedure for Preparing Silicone Shampoos from Premix Formulation I—Virgin European Medium Brown Hair

The same premix Formulation I was used to prepare shampoos for testing on virgin brown hair, however, the polymer concentration in the shampoo was 0.4 wt %, the amount of ammonium chloride used in these shampoos was 1.0 gram, and the amount of silicone used was 2.45 g GE SM555 dimethicanol.

Wet/Dry Comb Performance Measurement—Lightly Bleached European Medium Brown Hair Conditions:

Measured at constant temperature and humidity (72 deg. F. and 50% relative Humidity)

Equipment:

Instron 1122 (2-lb. load cell, 500-gram range used)

Procedure:

Each tress was washed twice with SLS using the standard washing/rinsing procedure.

The twice washed tress was hand combed 5-times with large teeth comb and 5-times with small teeth comb. (10× total)

No Instron testing of SLS-washed tresses

The washed tresses were allowed to sit overnight.

No dry-combing

1. Each tress was shampooed twice with the agreed upon shampoo amount. (0.5 g shampoo per 1 gram tress (all tresses were 3.0 g)

2. Each shampooed tress was hand combed twice with a large teeth comb.

3. The hand combed twice tress was loaded into a Instron instrument and the crosshead was lowered to bottom stop. The tress was combed twice with small teeth comb and placed into double-combs.

The Instron was run under standard conditions.

After the test was run, the tress was sprayed with DI water to keep moist. Do not hand-comb tress. Using a paper towel, wipe excess liquid off double-combs.

Return crosshead to bottom stop and replace tress into double-combs.

Rerun under standard conditions. A total of eight tests were run on each tress.

4. After the eight tests were finished, the tress was hung up overnight.

5. The next day, each tress was dry combed tested eight times. No hand combing of dry tresses was done.

6. Averaged wet comb energy for 40 Instron runs and reported average with standard deviation.

7. Averaged dry comb energy for 40 Instron runs and reported average with standard deviation.

A similar combing protocol was used for virgin hair, but only two tresses were used, and the average reported from the two tresses combed 5 times per tress, with more precombing of the tresses prior to measurement.

Several examples of the above technologies were demonstrated in the following Examples 1-6 in shampoo Formulation I using the standard combing protocol on bleached hair and virgin brown hair. This formulation is shown only for example and other formulations containing other silicones, or other oils, such as mineral oil or any other commonly used conditioning oil, humectants such as glycerol, or conditioning ingredients, such as panthenoic acid or derivatives can be included.

Measurement and Calculation of Alkyl Ether Content

The alkyl ether content of the substituted cellulose ethers shown in the examples is determined by reacting a sample with concentrated hydriodic acid at elevated temperature to produce alkyl iodides at temperatures of about 185 C for 2 hours. The reaction products are extracted in situ into a solvent (o-xylene) and the alkyl iodides are quantified by gas chromatography. This is the so called sealed tube Zeisel-GC technique. The amount of alkyl iodide produced by the sample is converted into the desired equivalent alkyl compound or functional group by multiplying by the ratio of molecular weights:

Species A×(mw B/mw A)=Species B

Specifically for cetyl content:

% cetyl iodide×mw cetyl/mw cetyl iodide=% cetyl

% cetyl iodide×225.45/3552.35=% cetyl

Molecular Weight

Weight average molecular weights were determined using aqueous size exclusion chromatography.

### Example 1

A gel of a water-soluble cetyl-modified hydroxyethyl cellulose (C16 HMHEC, 1.14 wt % cetyl substitution, 3.8 molar hydroxyethyl substitution, Mw=824,000 Dalton) that formed above 1.5-2 wt % polymer concentration and underwent syneresis upon dilution in water was used in this Example and showed very good efficacy in a 2-in-1 conditioning shampoo without the need for any cationic moiety and without depositing any silicone. For bleached hair, wet hair comb energy was reduced 30% relative to the wet comb energy for the no polymer control shampoo, and silicone deposition was less than 10 ppm. Wet comb energies for the shampoo containing the cationic guar benchmark, NHance® 3916 product, were reduced 40% relative to the no polymer shampoo.

This Example demonstrates that the nonionic hydrophobic polymer that undergoes syneresis in aqueous solution or in the shampoo on dilution can achieve nearly 75% of the wet comb energy reduction achieved by the cationic polymer. The dry comb energies for the tresses treated with a shampoo containing the polymers of the invention were equal to the dry comb energy measured on tresses treated with the shampoo containing no polymer and the shampoo containing cationic guar.

### Example 2

A water-soluble C16 HMHEC (1.04 wt % cetyl substitution, 4.0 molar hydroxyethyl substitution, Mw=1,200,000 Dalton) was used in this Example. This polymer formed a gel at 3-4 wt % polymer in water but showed syneresis at 2 wt %, was dissolved in 5 wt % ammonium lauryl sulfate to give a clear solution, and underwent syneresis upon dilution with water. This polymer showed very good efficacy in 2-in-1 conditioning shampoos without the need for any cationic moiety and without depositing any silicone. For bleached hair, wet hair comb energy was reduced by 28% relative to the no polymer control shampoo, and silicone deposition was less than 10 ppm. Wet hair comb energy reduction was 70% of the wet comb energy reduction achieved by cationic guar. The dry comb energies for the tresses treated with a shampoo containing the polymers of the invention were equal to the dry comb energy measured on tresses treated with the shampoo containing no polymer and the shampoo containing cationic guar.

### Example 3 (Comparative)

A shampoo was made with a water-soluble cetyl-modified hydroxyethyl cellulose (Polysurf® 67 product, 0.5 wt % cetyl substitution, 2.5 molar hydroxyethyl substitution, Mw=830,000 Dalton) that did not form a gel above 1.5-2 wt % polymer concentration and did not undergo syneresis upon dilution in water. For bleached hair, wet hair comb energy was reduced by 13% relative to the wet comb energy for the no polymer control shampoo, and silicone deposition was less than 10 ppm.

This Example demonstrates that the nonionic hydrophobic polymer that does not undergo syneresis does not show as good efficacy in the 2-in-1 conditioning shampoo as a polymer that undergoes dilution deposition (Examples 1-3). The dry comb energies for tresses treated with a shampoo containing the commercial Polysurf 67 product was equivalent, within standard deviation, of the dry comb energy measured on tresses treated with the shampoo containing no polymer and the shampoo containing cationic guar.

### Example 4 (Comparative)

A HMHEC polymer that was water-insoluble (2.82 wt % cetyl substitution, 3.83 molar hydroxyethyl substitution, dissolved with added surfactant in shampoo, yet did not undergo syneresis upon dilution and hence showed low efficacy in wet comb reduction. For bleached hair, wet hair comb energy was reduced by 11% relative to the wet comb energy for the no polymer control shampoo, and silicone deposition was less than 10 ppm. The dry comb energies for the tresses treated with a shampoo containing this polymer were equal to the dry comb energy measured on tresses treated with the shampoo containing no polymer and the shampoo containing cationic guar. This Example demonstrates that water-insolubility is not a defining criteria for performance, and syneresis of the water-insoluble polymer is required for performance.

### Example 5

A gel of a water-soluble methylphenylglycidyl hydroxyethyl cellulose ether, (6.3 wt % methylphenyl substitution, 2.5 molar hydroxyethyl substitution, Mw=350,000 Dalton), formed a gel above 1.5-2 wt % polymer concentration and underwent syneresis upon dilution in water and showed good efficacy in 2 in-1 conditioning shampoos without the need for any cationic moiety and depositing less than 30 ppm silicone. For virgin medium brown European hair, wet hair comb energy reduction was 72% of the wet comb energy reduction achieved by cationic guar. A silky feel was imparted to the hair.

Wet comb energy for the shampoo containing the cationic guar benchmark, NHance® 3916 product, was reduced 61% relative to the no polymer shampoo, with greater than 40 ppm silicone deposited. This Example demonstrated that the nonionic hydrophobic polymer that undergoes syneresis in aqueous solution or in the shampoo on dilution can achieve nearly 74% of the wet comb energy reduction achieved by the cationic polymer on virgin hair, with less silicone deposition. The dry comb energies for the tresses treated with a shampoo containing the polymer of the invention were equal to the dry comb energy measured on tresses treated with the shampoo containing no polymer and the shampoo containing cationic guar.

### Examples 6-28

Simple conditioning tests were performed evaluating polymers of the invention and some commercial polymers on mildly bleached hair using a fully formulated rinse-off conditioner (Examples 6-16) and aqueous solutions of the polymers (Examples 17-28). The Instron comb test described below was used to generate the data shown in these Examples. Comparison of the wet and dry comb energy Example 16 with other Examples in the Table demonstrated that the polymer of the invention delivered the lowest combined wet and dry comb energies of all nonionic and hydrophobic polymers tested and approached the wet and dry comb energies delivered by cationic polymers of Example 8. In Table 2, comparison of the wet and dry comb energy Example 28 with other examples in the Table 2 demonstrated that the polymer of the invention delivered the lowest combined wet and dry comb energies of all nonionic and hydrophobic polymers tested and approached the wet and dry comb energies delivered by cationic polymers of Examples 18-20.

### Polymers as a Conditioner in Fully Formulated Conditioning Formulation—Table 1

Natrosol® hydroxyethyl cellulose type 250HHR was added to water under agitation. Next, pH was adjusted to 8.0 to 8.5. The slurry was stirred for about 30 minutes or until polymer dissolved. Next, polymer of this invention or a commercial comparative polymer listed in TABLE 1 was added and mixed for 30 more minutes. The solution was heated to about 65° C. and stirred until it became smooth. Cetyl alcohol was added and mixed until it mixed homogeneously. The mixture was cooled to about 50° C. and then potassium chloride was added. Next, isopropyl myristate was added and mixed until the mixture looked homogeneous. The pH of the mixture was adjusted between 5.25 to 5.5 with citric acid and/or NaOH solution. The conditioner was preserved with 0.5% preservative and mixed until it reached room temperature.

| | |
|---|---|
| 90.94 g | Deionized water |
| 00.70 g | Natrosol ® 250HHR |
| 00.20 g | Polymer of this invention or commercial polymer |
| 02.00 g | Cetyl alcohol |
| 00.50 g | Potassium Chloride |
| 02.00 g | Isopropyl Palmitate |
| As required | Citric acid to adjust pH |
| As required | Sodium hydroxide to adjust pH |
| 00.50 g | Preservative |

About three grams in weight flat tresses of mildly bleached European hair from International Hair Importers and Products Inc. of Glendale, N.Y. were used for measuring wet and dry combing performance of various formulations of this experiment. To clean the hair tress, the hair tress was first wetted with 40° C. tap water and then 5.0 ml of sodium lauryl sulfate solution was applied along the tress length. Tress was kneaded for 30 second. Tress was then rinsed under 40° C. running water for 30 seconds followed by rinsing with room temperature tap water for 30 seconds. The tress was then dried overnight. Next day, the tress was rewetted with 40° C. tap water. Next, 0.5 gram of test conditioner per gram of hair was applied uniformly along the length of hair. Tress was kneaded for 30 second and then it was rinsed under 40° C. running water for 30 seconds. The conditioner was reapplied along the length of the tress and the tress was kneaded for 30 second; then, it was rinsed under 40° C. running water for 30 seconds. The tress was rinsed with room temperature tap water for 30 seconds. The tress was combed immediately eight times and from the data average amount combing energy in gram force-mm/gram of hair (gf-mm/g) required to comb the hair was calculated. The tress was stored overnight at about 50% relative humidity and about 23° C. Next day, the tress was first combed with fine teeth rubber comb to free-up hair stuck together. Again, the hair tress was combed eight times to determine the average force required to comb one gram of dry hair. The higher the number the poorer the conditioning effect of the polymer being tested. Two tresses were used per conditioning formulation. The data reported below are average of two tresses.

TABLE 1

| | | | | | Conditioner | Wet | Dry | |
|---|---|---|---|---|---|---|---|---|
| | Polymer of | Comparative | Polymer | Polymer | Viscosity | Combing | Combing | |
| Example# | Invention | Polymer | type | Level/wt % | (cps) | (gf-mm/g) | (gf-mm/g) | Comments |
| 6 | | Polymer-free Control | — | 0 | 990 | 4774 | 287 | Stable |
| 7 | | Polymer-Free Control | — | 0 | 1380 | 4513 | 364 | Stable |
| 8 | | N-Hance ® 3269 | cationic | 0.2 | 1330 | 1389 | 263 | Stable |
| 9 | | Natrosol ® 250HHR | nonionic | 0.2 | 1970 | 4320 | 361 | Stable |
| 10 | | Natrosol 250HHR | nonionic | 0.2 | 2100 | 2700 | 290 | Stable |
| 11 | | UCARE ® LR400 | cationic | 0.2 | 1280 | 811 | 1116 | Stable |
| 12 | | Nexton ® 3082R | hydrophobic | 0.2 | 2280 | 4941 | 312 | Stable |
| 13 | | Natrosol ® Plus 330 | hydrophobic | 0.2 | 1670 | 2565 | 340 | Stable |
| 14 | | Polysurf 67 | hydrophobic | 0.2 | 2170 | 2952 | 459 | Stable |
| 15 | | AQU D3673 | hydrophobic | 0.2 | 1080 | 2281 | 625 | Stable |
| 16 | AQU D3930 | | hydrophobic | 0.2 | 1940 | 2262 | 298 | Stable |

Ingredient List FOR TABLE 1:
(1) Natrosol ® 250HHR: Hydroxyethyl cellulose from Hercules, Inc. Wilmington, DE
(2) Nexton ® 3082R: C4 hydrophobically modified hydroxyethyl cellulose from Hercules, Inc. Wilmington, DE
(3) Polysurf ® 67:, NT4C3594, C16 hydrophobically modified hydroxyethyl cellulose from Hercules, Inc.
(4) Natrosol Plus 330: NT43669, C16 hydrophobically modified hydroxyethyl cellulose from Hercules, Inc.
(5) UCARE LR400:, Cationic HEC from Dow Chemicals, Midland, MI
(6) UCARE JR30M:, Cationic HEC from Dow Chemicals, Midland, MI
(7) N-Hance ® 3269: cationic guar cationic DS 0.13, Weight average Molecular weight 500,000 from Hercules Inc. Wilmington, DE
(8) AquaCat ® CG 518: cationic guar, cationic DS 0.18, Weight average Molecular weight 50,000 from Hercules Inc. Wilmington, DE
(9 AQU D3930:, Polymer of this invention, C16 hydrophobically modified hydroxyethyl cellulose from Hercules, Incorporated 0.62 wt % cetyl, hydroxethyl molar substitution(HEMS) 4.0
(10) AQU D3673:, C8 hydrophobically modified hydroxyethyl cellulose from Hercules, Inc.
(11) Crodacol C95NF: Cetyl alcohol from Croda Inc. Parsippany, NJ
(112KCI: Potassium chloride
(13) Stepan IPM: Isopropyl myristate from Stepan Company, Northfield, IL
(14) Germaben II: preservative from ISP Wayne, NJ

### Polymers as a Detangling Agent/Conditioning Agent in Aqueous System

### Table 2

Polymers of this invention or comparative polymers, listed in Table 2, were added to water under agitation to form a slurry. Next, pH was adjusted to 8.0 to 8.5 for cellulosic polymers and to about 6.5 for guar based products. The slurry was mixed for about 60 minutes or until the polymer fully dissolved. Then, the pH of the mixture was adjusted to between 5.25 to 5.5 with citric acid and/or NaOH solution. The conditioner was preserved with 0.1% preservative and mixed for 15 minutes. The pH was readjusted as necessary.

Ingredients:

| | |
|---|---|
| 99.70 g | Deionized water |
| 00.20 g | Polymer of this invention or commercial polymer |
| As required | Citric acid to adjust pH |
| As required | Sodium hydroxide to adjust pH |
| 00.10 g | Preservative |

About three grams in weight of flat tresses of mildly bleached European hair from International Hair Importers and Products Inc. of Glendale, N.Y. were used for measuring wet and dry combing performance of various formulations of this Example. To clean the hair tress, the hair tress was first wetted with 40° C. tap water and then 5.0 ml of sodium lauryl sulfate solution was applied along the tress length. The tress was kneaded for 30 second. The tress was then rinsed under 40° C. running water for 30 seconds' followed by rinsing with room temperature tap water for 30 seconds. The tress was then dried overnight. Next day, the tress was rewetted with 40° C. tap water. Next, 0.5 gram of test solution per gram of hair was applied uniformly along the length of hair. The tress was kneaded for 30 second and then was rinsed under 40° C. running water for 30 seconds. The test solution was reapplied along the length of the tress and the tress was kneaded for 30 second and then was rinsed under 40° C. running water for 30 seconds. The tress was rinsed with room temperature tap water for 30 seconds. The tress was combed immediately eight times to calculate the average amount of combing energy in gram force-mm/gram of hair (gf-mm/g) required to comb the hair. The tress was stored overnight at about 50% relative humidity and about 23° C. Next day, the tress was first combed with fine teeth rubber comb to free-up hair stuck together. Again, hair tress was combed eight times to determine average force required to comb one gram of dry hair. The higher the number the poorer the conditioning effect of the polymer being tested. Two tresses were used per conditioning formulation. Combing data below are average of two tresses.

TABLE 2

| | Polymer | | | | Wet | Dry |
|---|---|---|---|---|---|---|
| | of | | | | Combing | Combing |
| Example# | Invention | Polymer Type | Comparative Polymer | Lot# | (gf-mm/g) | (gf-mm/g |
| 17 | | — | Polymer-free Control | | 5267 | 318 |
| 18 | | Cationic | N-Hance ® 3269 | | 1553 | 497 |
| 190 | | Cationic | AquaCat ® CG518 | | 1123 | 185 |
| 201 | | Cationic | N-Hance ® 3196 | | 1830 | 659 |
| 212 | | Nonionic | Natrosol ® 250HHR | | 2811 | 314 |
| 22 | | Cationic | UCARE ® LR400 | | 607 | 515 |
| 23 | | Cationic | UCARE ® JR30M | | 759 | 334 |
| 24 | | Hydrophobic | Nexton ® 3082R | | 5631 | 410 |
| 25 | | Hydrophobic | Nexton J20R | | 5774 | 434 |
| 26 | | Hydrophobic | Natrosol ® Plus 330 | | 2059 | 333 |
| 27 | | Hydrophobic | Polysurf 67 | | 2451 | 451 |
| 28 | AQU | Hydrophobic | | | 1798 | 463 |
| | D3930 | | | | | |

Ingredient List FOR TABLE 2:
(1) Natrosol ® 250HHR: Hydroxyethyl cellulose from Hercules, Inc. Wilmington, DE
(2) Nexton ® 3082R: C4 hydrophobically modified hydroxyethyl cellulose from Hercules, Inc., Wilmington, DE
(3) Nexton ® J20R, C4hydrophobically modified hydroxyethyl cellulose from Hercules, Inc. Wilmington, DE
(4) Polysurf ® 67: NT4C3594, C16 hydrophobically modified hydroxyethyl cellulose from Hercules, Inc.
(5) Natrosol Plus 330: NT43669, C16 hydrophobically modified hydroxyethyl cellulose from Hercules, Inc.
(6) UCARE LR400: Cationic HEC from Dow Chemicals, Midland, MI
(7) UCARE JR30M: Cationic HEC from Dow Chemicals, Midland, MI
(8) N-Hance ® 3269: cationic guar cationic DS 0.13, Weight average Molecular weight 500,000 from Hercules Inc. Wilmington, DE
(9) N-Hance ® 3196: cationic guar cationic DS 0.13, Weight average Molecular weight 1.2 MM from Hercules Inc. Wilmington, DE
(10) AquaCat ® CG 518: cationic guar, cationic DS 0.18, Weight average Molecular weight 50,000 from Hercules Inc. Wilmington, DE
(11) AQU D3930: Polymer of this invention, C16 hydrophobically modified hydroxyethyl cellulose from Hercules, Inc. 0.62 wt % cetyl, hydroxethyl molar substitution(HEMS) 4.0
(12) Kathon CG: Preservative from Rohm & Haas

### Examples 29-39

A skin lotion was prepared containing the polymer of the invention (Example 33) and compared with a polymer-free skin lotion (Example 30), skin lotions containing hydrophobic polymers which did not undergo syneresis (Examples 32, 36, 40) and with skin lotions containing commercial nonionic and cationic polymers. The skin lotion containing the polymer of the invention showed increased viscosity and structure as compared with the polymer-free control formulation in Example 30; Example 33 was more stable than the formulations containing cationic polymer. Compared with the commercial hydrophobic polymers, the polymer of the invention appeared slightly grainy, suggesting that this polymer could be used at a lower concentration than commercial hydrophobic polymers.

### Fully Formulated Skin Lotion—Single Polymer—Table 3

| Ingredient | | Weight % Active |
|---|---|---|
| A. | Polymer | 0.50 |
| | Distilled water | 78.00 |
| | Glycerin, | 2.00 |
| B. | Glycol stearate (Kessco ® EGMS) | 2.75 |
| | Stearic acid (Industrene ® 5016) | 2.50 |
| | Mineral oil (Drakeol ® 7) | 2.00 |
| | Acetylated lanolin (Lipolan ® 98) | 0.50 |
| | Cetyl alcohol (Crodacol ® C95) | 0.25 |
| C. | Distilled water | 10.00 |
| | Triethanolamine | 0.50 |
| D. | Propylene glycol and diazolidinyl urea | 0.75 |
| | and methylparaben and propylparaben | |
| | (Germaben II) | |
| | | 100.00 |

Procedure:

Polymer listed in Table 3 was dispersed in water by adding to the vortex of well-agitated from Part A. It was mixed for five minutes. Next, glycerin was added with continued mixing and heated to 80° C. Mixed 15 minutes at 80° C. In a separate vessel, blended Part B ingredients and heated to 80° C. and mixed well.

Part A was added to Part B with good agitation while maintaining emulsion temperature at 80° C. Part C ingredients were mixed together in a vessel and added to the emulsion of Parts A and B. The new mixture was mixed continuously while cooling to 40° C. Then, the pH was adjusted to between 6.0 to 6.5. Then Part D (preservative) was added to the emulsion and mixed well. The new emulsion was then cooled and filled.

TABLE 3

| | Polymer of | | Commercial | Lotion Viscosity | | |
|---|---|---|---|---|---|---|
| Example# | Invention | Polymer Type | Polymer | at 5 rpm | pH | Comments |
| 30 | | — | Control - Polymer-Free | 6800 | 6.3 | Fluid |
| 31 | | hydrophobic | Natrosol ® Plus 330 | 124,000 | 6.2 | Smooth, Glossy, cream |
| 32 | | cationic | N-Hance ® 3215 | | | Phase separation |
| 33 | AQU D3930 | hydrophobic | | 164,000 | 6.4 | Stable, grainy, Highly structured |
| 34 | | cationic | UCARE ® LR400 | 28000 | 6.2 | Curdled appearance. No separation |
| 35 | | cationic | UCARE ® JR30M | 19200 | 6.1 | Curdled appearance. No separation |
| 36 | | hydrophobic | Polysurf 67 | 165,000 | 6.4 | Stable, glossy, Highly structured |
| 37 | | nonionic | Natrosol 250M | 5600 | 6.3 | FluidGlossy |
| 38 | | nonionic | Natrosol 250LR | 4400 | 6.6 | Fluid Glossy |
| 39 | | hydrophobic | AQU D3673A | 10800 | 6.5 | Fluid, Glossy |
| 40 | | hydrophobic | Nexton 3082R | | | |

Ingredient List FOR TABLE 3:
(1) Kessco ® EGMS: Stepan Company, Northfield, IL
(2) Inustrene ® 5016: Crompton Corp. Middleburry, CT
(3) Drakeol ® 7: Penreco, Pennzoil Products Company Karn City, PA
(4) Lipolan 98: Lipo Chemicals. Inc. Paterson, NJ
(5) Crodacol ® C95: Croda IncParsippany, NJ
(6) Germaben II: preservative from ISP Wayne, NJ
(7) Natrosol ® Plus 330: C16 Hydrophobically modified Hydroxyethyl cellulose Hercules Inc. Wilmington, DE
(8) N-Hance 3215: Cationic guar, Hercules Inc. Wilmington, DE
(9) AQU D3930:; Polymer of this invention, C16 hydrophobically modified hydroxyethyl cellulose from Hercules, Inc. 0.62 wt % cetyl, hydroxethyl molar substitution(HEMS) 4.0
(10) UCARE LR400: Cationic HEC from Dow Chemicals, Midland, MI
(11) UCARE JR30M: Cationic HEC from Dow Chemicals, Midland, MI
(12) Polysurf ® 67: NT4C3594, hydrophobically modified hydroxyethyl cellulose from Hercules, Inc.
(13) Natrosol ® 250LR: lot#28667, Hydroxyethyl cellulose from Hercules, Inc. Wilmington, DE
(14) Natrosol ® 250M: Hydroxyethyl cellulose from Hercules, Inc. Wilmington, DE
(15) Nexton ® 3082RC4 hydrophobically modified hydroxyethyl cellulose from Hercules, Inc. Wilmington, DE
(16) Natrosol 250HHR CS, Hydroxyethyl cellulose from Hercules, Inc. Wilmington, DE
(17) AQU D3673: C8 hydrophobically modified hydroxyethyl cellulose from Hercules, Inc.

### Examples 41-51

A body wash formulation was prepared using the polymer of the invention (Example 43) with a polymer-free control (Example 41) and with formulations containing commercial nonionic, hydrophobic, and cationic polymers. The polymer of the invention (Example 43) showed better compatibility with the body wash components than the nonionic commercial polymers (Examples 48 and 50). The commercial hydrophobic polymers conveyed an applesauce texture to the formulation, as did the polymer of the invention. This result suggests that these polymers could be used at a lower concentration in this formulation.

### Body Wash Table 4

Body wash preparation: An aqueous stock solution of each polymer was first prepared at 1.0% concentration. For polymers: N-Hance® 3215, ADPP6503, AQU D3799, and AQU D3939 solutions were made by adding polymer to water under vigorous agitation. Next, the pH was lowered to between 6 to 7 with citric acid and the solution was mixed for an hour or until the polymer solubilized. The solutions were preserved with 0.5% Glydant® product. For the polymers ADPP6531, ADPP5922, AQU D3869, AQU D3673, ADPP6582 ADPP6626, Polysurf® 67, Natrosol® plus 330, Natrosol® 250HHR, Natrosol® 250M, UCARE® JR30M, UCARE® JR400, AQU D3686 ADPP6641, the polymers were added to well agitated water and then the pH was raised to 8.5 to 9.5 using sodium hydroxide. The solution was mixed for an hour and then the pH was lowered to between 6 to 7 using citric acid.

Body wash stock solution was prepared by adding to vessel 46.4 grams of sodium laureth sulfate, 27.0 grams of sodium lauryl sulfate, 6.7 grams of C₉-C₁₅ alkyl phosphate, 4.0 grams of PPG-2 hydroxyethyl cocamide, 1.0 gram of sodium chloride, 0.30 gram of tetra sodium EDTA, and 0.5 gram of DMDM hydantoin in the order listed while mixing. Each ingredient was allowed to mix homogeneously before adding the next ingredient. The total stock solution weighed 85.9 grams.

Body wash was prepared by adding 20 grams of polymer (listed in Table 4) solution to 80 grams of the above body wash stock solution while mixing. Next, the body wash pH was adjusted to between 6 and 7 with citric acid. The body wash viscosity was measured using the Brookfield LVT viscometer. The viscosity was measured at 30 rpm once the body wash conditioned for at least two hours at 25° C. The body wash clarity was also measured at 600 nm using a Spectrophotometer, Cary 5E UV-VIS-NIR, available from Varian Instruments, Inc. The clarity measurements at 600 nm wavelength are reported as % T value. The higher the number, the clearer is the solution.

Lather Drainage Test:

Objective of this Test is to measure the lather drainage time of a diluted body wash solution. Long drainage times indicate a rich, dense lather with good stability. The Test was used to determine the influence that the polymers of this invention may have on lather quality.

Equipment:

Waring® Blender Model #7012 or 34BL97 or equivalent.

Funnel, preferably plastic; 6″ diameter, ⅞″ ID neck, 5¼″ high, with a horizontal wire 2″ from the top.

U.S.A. Standard Testing Sieve NO.20 or Tyler® Equivalent 20 mesh or 850 micrometer or 0.0331 inch sieve. Preferably over 7 inch in diameter but smaller size could also be used

Stopwatch or a timer.

Procedure:

For each test formulation, 1,000 g of a diluted body wash solution was prepared as shown below.

| | | |
|---|---|---|
| Body wash | 66.13 | g |
| Deionized Water | 933.87 | g |
| Total | 1,000.00 | g |

1. For each lather test measurement 200 grams of above diluted solution was weighed and placed in a 25° C. water-bath for 2 hours. Three jars (each with 200 grams of solution) were prepared per body wash formulation.
2. Next, the lather drainage time for each solution was measured using the procedure described below.

a. 200 g of solution were poured into a clean, dry Waring blender glass vessel.

b. The solution was blended at the highest speed for exactly 1 minute while covered.

c. Foam generated in the jar was immediately poured into a clean, dry funnel standing on a 20 mesh screen over a beaker.

d. Foam from the blender was poured for exactly 15 seconds. The goal was to get as much foam as possible into the funnel without overflowing. After 15 seconds, stopped pouring foam, however, the stopwatch was kept running.

e. The total time needed for the foam to drain including the 15 seconds for pour time was recorded once the wire was no longer covered by foam or liquid.

TABLE 4

| | | | | | Lather | | |
|---|---|---|---|---|---|---|---|
| | Polymer of | | | Visc. | Stability | T | |
| Example# | Invention | Polymer Type | Commercial Polymer | cps | Seconds | (%) | Comments |
| 41 | | — | Control - Polymer-Free | 3680 | 54 | 99.4 | |
| 42 | | Cationic | N-Hance ® 3215 | 6100 | 98.7 | 85.9 | |
| 43 | AQU D3930 | Hydrophobic | | 3960 | 57.3 | 25.2 | Applesauce like structure, separation |
| 44 | | Cationic | UCARE ® JR400 | 6420 | 52.7 | 78.8 | |
| 45 | | Cationic | UCARE ® JR30M | 19120 | 57.5 | 98.5 | |
| 46 | | Hydrophobic | Natrosol ® Plus 330 | 4080 | 64.3 | 21.6 | Applesauce like structure |
| 47 | | Hydrophobic | Polysurf 67 | 4080 | 52.3 | 14.2 | Applesauce like structure |
| 48 | | Nonionic | Natrosol 250M | 4540 | Not Run | 32.4 | Gels - incompatible |
| 49 | | Hydrophobic | Nexton 3082R | 4420 | 53.3 | | |
| 50 | | Nonionic | Natrosol 250HHR CS | 4680 | Not run | 52.1 | Gels - Incompatible |
| 51 | | hydrophobic | AQU D3673A | 3560 | 60 | 95.5 | |

Ingredient List FOR TABLE 4:
(1) Sodium Lauryl sulfate - Stepanol ® WAC, Stepan Company Northfield, IL 60093.
(2) Sodium laureth Sulfate-Rhodapex ® ES-2, Rhodia, Cranbury, NJ 08512
(3) Cocamidopropyl betaine - Amphosol ® CA, Stepan Company Northfield, IL 60093.
(4) PPG-2 Hydroxyethyl Cocamide - Promidium ® CO, Uniqema, Newcastle, DE
(5) Tetra Sodium EDTA - Fisher Scientific.
(7) DMDM Hydantoin, Glydant ®, Lonza Inc. Fair Lawn, NJ, USA
(8) Sodium Chloride from Baker.
(9) Natrosol ® Plus 330 - NT3J3314, C16 Hydrophobically modified Hydroxyethyl cellulose Hercules Inc. Wilmington, DE
(10) N-Hance 3215: J4013A, Cationic guar, Hercules Inc. Wilmington, DE
(11) AQU D3930: Polymer of this invention, C16 hydrophobically modified hydroxyethyl cellulose from Hercules, Inc. 0.62 wt % cetyl, hydroxethyl molar substitution(HEMS) 4.0
(12) UCARE JR400: Cationic HEC from Dow Chemicals, Midland, MI
(13) UCARE JR30M: Cationic HEC from Dow Chemicals, Midland, MI
(14) Polysurf ® 67: NT4C3594, hydrophobically modified hydroxyethyl cellulose from Hercules, Inc.
(15) Natrosol ® 250M: Hydroxyethyl cellulose from Hercules, Inc. Wilmington, DE
(16) Nexton ® 3082R: hydrophobically modified hydroxyethyl cellulose from Hercules, Inc. Wilmington, DE
(17) Natrosol 250HHR CS, Hydroxyethyl cellulose from Hercules, Inc. Wilmington, DE
(18) AQU D3673: C8hydrophobically modified hydroxyethyl cellulose from Hercules, Inc.

### Examples 52-62

The polymer of the invention was incorporated into a sunscreen formulation. (Example 54). The formulation was stable.

### Sunscreen Lotion Table 5

The Drakeol mineral oil was heated in a vessel to 75° C. while mixing. Next, the remaining ingredients of Part A (Arlmol E, Neo Heliopan AV, Uvinol M40, Castor wax, Crill-6, Arlatone T, Ozokerite wax and Dehymuls HRE7) were added to the vessel in the order listed while mixing. The mixture was mixed for 30 minutes at 70° C. In a separate container water of Part B was heated to 70 C. Next, the polymer of invention or comparative polymer (listed in Table 5) was added and mixed until dissolved and then Glycerine was added and mixed. In a separate container a solution of magnesium sulfate was prepared by adding magnesium sulfate to water. Next, the solution of magnesium sulfate was added to Part B and mixed until heated back to 70° C. This mixture was then added to Part A while mixing And then mixed for 30 minutes at 70° C. and then cooled to room temperature while mixing. Preservative Germaben II was added when temperature reached below 50° C.

Part A

| | |
|---|---|
| 13.0 g | Drakeol 7: Mineral oil. |
| 6.0 g | Arlamol E: PPG-15 Stearyl ether |
| 1.0 g | Neo Heliopan AV: Octyl methoxcinnamate |
| 1.0 g | Uvinol M40: Benzophenone-3 |
| 1.4 g | Castor Wax: Hydrogenated castor oil |
| 1.2 g | Crill-6: Sorbitan iostearate |
| 1.0 g | Arlatone T: PPG-40 Sorbitan Peroleate |
| 1.0 g | Ozokerite Wax 77W: Wax |
| 0.5 g | Dehymuls HRE7: PEG-7 hydrogenated castor oil |

Part B

| | |
|---|---|
| 40.5 g | Deionized water |
| 0.5 g | Polymer |
| 3.0 g | Glycerine |

Part C

| | |
|---|---|
| 23.1 g | Deionized water |
| 0.7 g | Magnesium Sulfate |

Part D

| | |
|---|---|
| 0.5 g | Germaben II - Preservative, |

TABLE 5

| | Polymer | | | | |
|---|---|---|---|---|---|
| | of | Polymer | Commercial | Visc. | |
| Example# | Invention | Type | Polymer | cps | Comments |
| 52 | | | Control -Polymer- | 4400 | |
| | | | Free | | |
| 53 | | | N-Hance ® 3215 | 2440 | |
| 54 | AQU | | | 6060 | |
| | D3930 | | | | |
| 55 | | | UCARE ® JR400 | 8120 | |
| 56 | | | UCARE ® JR30M | 3516 | |
| 57 | | | Natrosol ® Plus | 5880 | |
| | | | 330 | | |
| 58 | | | Polysurf 67 | 5260 | |
| 59 | | | Natrosol 250M | 3540 | |
| 60 | | | Nexton 3082R | 5700 | |
| 61 | | | Natrosol 250HHR | 2500 | |
| | | | CS | | |
| 62 | | | AQU D3673A | | Phase |
| | | | | | separation |

Ingredient List FOR TABLE 5:
(1) Drakeol 7: Mineral oil, Penereco, Karn City, PA.
(2) Arlamol E: OOG-15 Stearyl ether, Uniqema Americas, New Castle, DE
(3) Neo Heliopan AV: Octyl methoxcinnamate, Symrise, Totowa, NJ
(4) Uvinol M40: Benzophenone-3, BASF, Mount Olive, NJ
(5) Castor Wax: Hydrogenated castor oil, Frank B. Ross
(7) Crill-6: Sorbitan iostearate, Croda Inc Parsippany, NJ
(8) Arlatone T: PPG-40 Sorbitan Peroleate, Uniqema Americas, New Castle, DE
(9) Ozokerite Wax 77W: Wax, Frank B. Ross
(10) Dehymuls HRE7: PEG-7 hydrogenated castor oil, Cognis, Amber, PA
(11) Magnesium sulfate - J. T. Baker, Phillpsburg, NJ
(12) Glycerine: Spectrum Bulk Chemicals, New Brunswick, NJ
(13) Germaben II - Preservative, ISP, Wayne, NJ
(14) Natrosol ® Plus 330 - NT3J3314, C16 Hydrophobically modified Hydroxyethyl cellulose Hercules Inc. Wilmington, DE
(15) N-Hance 3215 - J4013A, Cationic guar, Hercules Inc. Wilmington, DE
(16) AQU D3930: Polymer of this invention, C16 hydrophobically modified hydroxyethyl cellulose from Hercules, Inc. 0.62 wt % cetyl, hydroxethyl molar substitution(HEMS) 4.0
(17) UCARE JR400: Cationic HEC from Dow Chemicals, Midland, MI
(18) UCARE JR30M: Cationic HEC from Dow Chemicals, Midland, MI
(19) Polysurf ® 67: NT4C3594, hydrophobically modified hydroxyethyl cellulose from Hercules, Inc.
(20) Natrosol ® 250M: Hydroxyethyl cellulose from Hercules, Inc. Wilmington, DE
(21) Nexton ® 3082R: hydrophobically modified hydroxyethyl cellulose from Hercules, Inc. Wilmington, DE
(22) Natrosol 250HHR CS, Hydroxyethyl cellulose from Hercules, Inc. Wilmington, DE
(23) AQU D3673: 11750-46, C8hydrophobically modified hydroxyethyl cellulose from Hercules, Inc.

### Examples 63-73

The polymer of the invention was incorporated into a roll-on antiperspirant formulation which was stable. (Example 65)

### Roll-On Antiperspirant

### Table 6

Antiperspirant preparation: An aqueous stock solution of each polymer was first prepared at 1.0% concentration. For polymers (N-Hance® 3215, ADPP6503, AQU D3799, and AQU D3939), solutions were made by adding the polymer to water under vigorous agitation. Next, the pH was lowered to between 6 to 7 with citric acid and the solution was mixed for an hour or until polymer solubilized. The solutions were preserved with 0.5% Glydant® product. For the polymers ADPP6531, ADPP5922, AQU D3869, AQU D3673, ADPP6582 ADPP6626, Polysurf® 67, Natrosol® plus 330, Natrosol® 250HHR, Natrosol® 250M, UCARE® JR30M, UCARE® JR400, AQU D3686 ADPP6641, the polymer was added to intensely agitated water and then the pH was raised to between 8.5 to 9.5 using sodium hydroxide. The solution was mixed for an hour and then the pH was lowered to between 6 to 7 using citric acid.

A 150 gram batch of roll-on antiperspirant was made using the procedure outlined below

15.0 g of Polymer (Listed in Table 6) were added to stock solution in an 8 oz glass jar and mixed with a magnetic plate and stirrer.

Next, 22.5 g of deionized water were add to the glass jar and mixing was continued for about 30 minutes. While mixing, 45.0 g of ethanol was added and the mixing was continued for an additional 10 minutes.

Then, 67.5 g of the antiperspirant active Summit ACH303 was added and the mixing was continued for 30 more minutes.

TABLE 6

| | Polymer of | | Visc. | |
|---|---|---|---|---|
| Example# | Invention | Commercial Polymer | cps | Comments |
| 63 | | Control - Polymer-Free | | Clear, water-white |
| 64 | | N-Hance ® 3215 | | Very Hazy, gels through-out |
| 65 | AQU D3930 | | | |
| 66 | | UCARE ® JR400 | | |
| 67 | | UCARE ® JR30M | | |
| 68 | | Natrosol ® Plus 330 | | Clear, water-white, fine particles through-out |
| 69 | | Polysurf 67 | | Clear, Trace haze, fine particles through-out |
| 70 | | Natrosol 250M | | Clear, water-white, fine particles through-out |
| 71 | | Nexton 3082R | | |
| 72 | | Natrosol 250HHR CS | | Clear, water-white, fine particles through-out |
| 73 | | AQU D3673A | | |

Ingredient List FOR TABLE 6:
(1) Ethanol: Dehydrated ethanol; Spectrum Chemicals MFG Corp, Gardena, CA.
(2) Summit ACH-303 - 50% aqueous solution of Aluminum Chlorohydrate, Summit Research Labs, 45 River Road, Flemington, NJ
(3) Natrosol ® Plus 330 -, NT3J3314, C16 Hydrophobically modified Hydroxyethyl cellulose Hercules Inc. Wilmington, DE
(4) N-Hance 3215: J4013A, Cationic guar, Hercules Inc. Wilmington, DE
(5) AQU D3673: 11750-46; Polymer of this invention, C8hydrophobically modified hydroxyethyl cellulose from Hercules, Inc.
(6) AQU D3930: Polymer of this invention, C16 hydrophobically modified hydroxyethyl cellulose from Hercules, Inc. 0.62 wt % cetyl, hydroxethyl molar substitution(HEMS) 4.0
(7) UCARE JR400: Cationic HEC from Dow Chemicals, Midland, MI
(8) UCARE JR30M: Cationic HEC from Dow Chemicals, Midland, MI
(9) Polysurf ® 67: NT4C3594, hydrophobically modified hydroxyethyl cellulose from Hercules, Inc.
(10) Natrosol ® 250M: Hydroxyethyl cellulose from Hercules, Inc. Wilmington, DE
(11) Nexton ® 3082R: hydrophobically modified hydroxyethyl cellulose from Hercules, Inc. Wilmington, DE
(12) Natrosol 250HHR CS, Hydroxyethyl cellulose from Hercules, Inc. Wilmington, DE

### Examples 74-81

The polymer of the invention was incorporated into Colgate-Palmolive Soft Body wash. The viscosity of the body wash increased (Example 77), and the y of the body wash was significantly better than for other commercial phobic cellulose ethers or nonionic cellulose ethers (Examples 78-81).

TABLE 7

| | Examples Soft Soap - 0.2% Active | | | | | | | | |
|---|---|---|---|---|---|---|---|---|---|
| | | | | Initial (24 hours) | | | | | |
| | | | | | Viscosity | Spindle# | | Solution | |
| Example | Designation | Source | Composition | pH | (cps) | rpm | % T | Clarity | pH |
| 74 | Control - 100 g of | | | 7.19 | 5060.0 | #4, 30 | 97.7 | Clear | 7.21 |
| | Soft Soap − no | | | | | | | | |
| | water or polymer | | | | | | | | |
| | added | | | | | | | | |
| 75 | Control - 80 g of | | | 7.20 | 175.0 | #2, 30 | 97.1 | Clear | 7.23 |
| | Soft Soap + 20 g of | | | | | | | | |
| | water added | | | | | | | | |
| 76 | AQU D3673 | Experimental | C8HMHEC | 7.14 | 337.0 | #2, 30 | 97.5 | Clear | 7.20 |
| 77 | AQU D3930 | polymer of | C16HMHEC | 7.17 | 1628.0 | #3, 30 | 87.4 | Very | 7.21 |
| | | invention | | | | | | slight | |
| | | | | | | | | hazy | |
| 78 | Polysurf 67 | Commercial | C16HMHEC | 7.04 | 1332.0 | #3, 30 | 32.5 | Very hazy | 7.17 |
| 79 | Nat. Plus 330 | Commercial | C16HMHEC | 7.09 | 783.0 | #2, 30 | 80.2 | Hazy | 7.15 |
| 80 | Natrosol 250HHR | Commercial | HEC | 7.11 | 249.0 | #2, 30 | 63.5 | Hazy | 7.17 |
| | CS | | | | | | (the | | |
| | | | | | | | polymer | | |
| | | | | | | | settled on | | |
| | | | | | | | the | | |
| | | | | | | | bottom, sample | | |
| | | | | | | | shaken | | |
| | | | | | | | before % T | | |
| | | | | | | | taken) | | |
| 81 | Natrosol 250M | Commercial | HEC | 7.11 | 236.0 | #2, 30 | 14.6 | Hazy | 7.18 |
| | | | | | | | (the | | |
| | | | | | | | polymer | | |
| | | | | | | | settled on | | |
| | | | | | | | the | | |
| | | | | | | | bottom, sample | | |
| | | | | | | | shaken | | |
| | | | | | | | before % | | |
| | | | | | | | taken) | | |

| | 2 weeks at room temp. | | | | |
|---|---|---|---|---|---|
| Example | Viscosity (cps) | Spindle# rpm | % T | Solution Clarity | Polymer Solubility |
| 74 | 4600.0 | #4, 30 | 97.5 | Clear | |
| 75 | 173.0 | #2, 30 | 97.1 | Clear | |
| 76 | 331.0 | #2, 30 | 96.8 | Clear | Soluble |
| 77 | 1736.0 | #3, 30 | 87.5 | Very slight hazy | Soluble |
| 78 | 1380.0 | #3, 30 | 40.4 | Very hazy | Soluble |
| 79 | 774.0 | #2, 30 | 81.2 | Hazy | Soluble |
| 80 | 282.0 | #2, 30 | 74.1 | Hazy | Polymer gel |
| | | | | | layer on bottom. |
| 81 | 282.0 | #2, 30 | 46.6 | Hazy | Polymer gel |
| | | | | | layer on bottom. |

Process:
1. Weigh 80 g commercial product into 4 oz. wide mouth glass jars.
2. Add 20 g of a 1% polymer solution.
3. Cap jars and tape lid with electrical tape. Shake by hand to initially mix polymer.
4. Place and secure jars on tumbler. Use tape across jars and around jars on ends to prevent from tumbling over edge.
5. Tumble jars far 1.5 hours. After 1.5 hours, remove jars and temper in 25 C. bath overnight.
6. After overnight, remove jars from bath, Observe and record solution clarity and polymer solubility. Take pH and viscosity, Measure % T at 600 nm for 24 hours sample. Store samples at ambient for 2 weeks and repeat temper in bath, observations, pH, viscosity, and % T.

### Examples 82-89

Incorporation of the polymer of the invention into Lysol All Purpose Cleaner, increased the product viscosity relative to the control product containing no polymer (Compare Example 85 with 82 in Table 8). The polymer of the invention was slow to dissolve in the Lysol base, but this could be improved with formulation optimization.

TABLE 8

| | Examples for Lysol All Purpose - Use at 0.2% Active | | | | | | | | |
|---|---|---|---|---|---|---|---|---|---|
| | | | | Initial (24 hours) | | | | | |
| | | | | | Viscosity | Spindle# | | Solution | Polymer |
| Example | Designation | | Composition | pH | (cps) | rpm | % T | Clarity | Solubility |
| 82 | Control - 100 g of | | | 8.78 | 4.1 | #1, 60 | 99.4 | Clear | Control |
| | Lysol − no water | | | | | | | | |
| | added | | | | | | | | |
| 83 | Control - 80 g of | | | 8.75 | 3.4 | #1, 60 | 99.2 | Clear | Control |
| | Lysol + 20 g of | | | | | | | | |
| | water added | | | | | | | | |
| 84 | AQU D3673 | Experimental | C8HMHEC | 8.57 | 4.2 | #1, 60 | 99.6 | Clear | Soluble |
| 85 | AQU D3930 | Polymer of | C16HMHEC | 8.62 | 10.5 | #1, 60 | 99.0 | Clear | insoluble, |
| | | Invention | | | | | | | undissolved |
| | | | | | | | | | polymer |
| 86 | Polysurf 67 | Commercial | C16HMHEC | 8.51 | 10.1 | #1, 60 | 98.4 | Clear | Soluble |
| 87 | Nat. Plus 330 | Commercial | C16HMHEC | 8.47 | 6.2 | #1, 60 | 99.2 | Clear | Soluble |
| 88 | Natrosol 250HHR CS | Commercial | HEC | 8.55 | 21.5 | #1, 60 | 99.0 | Clear | Soluble |
| 89 | Natrosol 250M | Commercial | HEC | 8.49 | 9.7 | #1, 60 | 99.6 | Clear | Soluble |

| | 2 weeks at room temp. | | | | | |
|---|---|---|---|---|---|---|
| Example | pH | Viscosity (cps) | Spindle# rpm | % T | Solution Clarity | Polymer Solubility |
| 82 | 8.79 | 3.50 | #1, 60 | 99.3 | Clear | Control |
| 83 | 8.79 | 3.20 | #1, 60 | 99.2 | Clear | Control |
| 84 | 8.68 | 4.40 | #1, 60 | 99.7 | Clear | Soluble |
| 85 | 8.64 | 11.30 | #1, 60 | 98.5 | Clear | Soluble |
| 86 | 8.58 | 12.40 | #1, 60 | 99.6 | Clear | Soluble |
| 87 | 8.55 | 6.00 | #1, 60 | 99.8 | Clear | Soluble |
| 88 | 8.62 | 19.10 | #1, 60 | 99.9 | Clear | Soluble |
| 89 | 8.55 | 11.10 | #1, 60 | 99.9 | Clear | Soluble |

Process:
1. Weigh 80 g commercial product into 4 oz. wide mouth glass jars.
2. Add 20 g of a 1% polymer solution.
3. Cap jars and tape lid with electrical tape. Shake by hand to initially mix polymer.
4. Place and secure jars on tumbler. Use tape across jars and around jars on ends to prevent from tumbling over edge.
5. Tumble jars for 1.5 hours. After 1.5 hours, remove jars and temper in 25 C. bath overnight.
6. After overnight, remove jars from bath. Observe and record solution clarity and polymer solubility. Take pH and viscosity, Measure % T at 600 nm. (24 hours sample) Store samples at ambient for 2 weeks and repeat temper in bath, observations, pH, viscosity, and % T.

### Examples 90-97

Incorporation of the polymer of the invention into Pinesol more than doubled the viscosity of the product. (Compare viscosity for Example 93 with 90 in Table 9).

TABLE 9

| | Examples for PineSol - Use at 0.2% Active | | | | | | | | |
|---|---|---|---|---|---|---|---|---|---|
| | | | | Initial (24 hours) | | | | | |
| | | Project | | | Viscosity | Spindle# | | Solution | Polymer |
| Example | Designation | Source | Composition | pH | (cps) | rpm | % T | Clarity | Solubility |
| 90 | Control - 100 g of | | | 10.1 | 43.0 | #2, 30 | 42.6 | Clear | Control |
| | Pinesol - no water | | | | | | | | |
| | added or polymer | | | | | | | | |
| | added | | | | | | | | |
| 91 | Control - 80 g of | | | 10.1 | 17.4 | #1, 30 | 50.5 | Clear | Control |
| | Pinesol + 20 g of | | | | | | | | |
| | water added | | | | | | | | |
| 92 | AQU D3673 | Experimental | C8HMHEC | 9.93 | 30.0 | #2, 30 | 50.2 | Clear | Soluble |
| 93 | AQU D3930 | Polymer of | C16HMHEC | 9.87 | 84.0 | #2, 30 | 49.3 | Very | Soluble |
| | | Invention | | | | | | slight | |
| | | | | | | | | hazy | |
| 94 | Polysurf 67 | Commercial | C16HMHEC | 9.85 | 78.0 | #2, 30 | 49.5 | Clear | Soluble |
| 95 | Nat. Plus 330 | Commercial | C16HMHEC | 9.85 | 40.0 | #1, 30 | 49.2 | Clear | Soluble |
| 96 | Natrosol 250HHR CS | Commercial | HEC | 9.86 | 143.0 | #2, 30 | 49.9 | Clear | Soluble |
| 97 | Natrosol 250M | Commercial | HEC | 9.88 | 75.0 | #2, 30 | 50.1 | Clear | Soluble |

| | 2 weeks at room temp. | | | | | |
|---|---|---|---|---|---|---|
| Example | pH | Viscosity (cps) | Spindle# rpm | % T | Solution Clarity | Polymer Solubility |
| 90 | 10.02 | 38.5 | #2, 30 | 42.3 | Clear | Control |
| 91 | 10.01 | 17.8 | #1, 30 | 50.5 | Clear | Control |
| 92 | 9.88 | 29.0 | #2, 30 | 50.3 | Clear | Soluble |
| 93 | 9.84 | 86.0 | #2, 30 | 48.4 | Very slight hazy | Soluble |
| 94 | 9.83 | 80.0 | #2, 30 | 49.9 | Clear | Soluble |
| 95 | 9.81 | 52.0 | #2, 30 | 49.8 | Clear | Soluble |
| 96 | 9.85 | 136.0 | #2, 30 | 50.3 | Clear | Soluble |
| 97 | 9.87 | 67.0 | #2, 30 | 50.5 | Clear | Soluble |

Process:
1. Weigh 80 g commercial product into 4 oz. wide mouth glass jars.
2. Add 20 g of a 1% polymer solution.
3. Cap jars and tape lid with electrical tape. Shake by hand to initially mix polymer.
4. Place and secure jars on tumbler. Use tape across jars and around jars on ends to prevent from tumbling over edge.
5. Tumble jars for 1.5 hours. After 1.5 hours, remove jars and temper in 25 C. bath overnight.
6. After overnight, remove jars from bath. Observe and record solution clarity and polymer solubility. Take pH and viscosity, measure % T at 600 nm. (24 hours sample) Store samples at ambient for 2 weeks and repeat temper in bath, observations, pH, viscosity, and % T.

### Examples 98-105

Incorporation of the product of the invention into Clorox (Compare Example 101 with 98) increased the viscosity of the product to a greater extent than any of the commercial hydrophobic or nonionic cellulose ethers in Table 10.

TABLE 10

| | Examples for Clorox - Use at 0.2% Active | | | | | | | | |
|---|---|---|---|---|---|---|---|---|---|
| | | | | Initial (24 hours) | | | | | |
| | 1% Solution | | | | Viscosity | Spindle# | | Solution | Polymer |
| Example | NB # X33892- | Designation | Composition | pH | (cps) | rpm | % T | Clarity | Solubility |
| 98 | Control - 100 g of | | | 3.44 | 55.1 | #1, 60 | 96.5 | Clear | Control |
| | bleach − no water | | | | | | | | |
| | added | | | | | | | | |
| 99 | Control - 80 g of | | | 3.51 | 10.6 | #1, 60 | 96.6 | Clear | Control |
| | bleach + 20 g of | | | | | | | | |
| | water added | | | | | | | | |
| 100 | AQU D3673 | experimental | C8HMHEC | 3.75 | 28.8 | #1, 60 | 95.6 | Clear | Soluble |
| 101 | AQU D3930 | Polymer of | C16HMHEC | 3.54 | 96.2 | #1, 30 | 95.8 | Clear | Soluble |
| | | Invention | | | | | | | |
| 102 | Polysurf 67 | Commercial | C16HMHEC | 3.61 | 81.2 | #1, 60 | 94.9 | Clear | Soluble |
| 103 | Nat. Plus 330 | Commercial | C16HMHEC | 3.63 | 31.9 | #1, 60 | 94.5 | Clear | Soluble |
| 104 | Natrosol 250HHR CS | Commercial | HEC | 3.55 | 79.6 | #1, 60 | 95.2 | Clear | Soluble |
| 105 | Natrosol 250M | Commercial | HEC | 3.56 | 34.1 | #1, 60 | 95.7 | Clear | Soluble |

| | 2 weeks at room temp. | | | | | |
|---|---|---|---|---|---|---|
| Example | pH | Viscosity (cps) | Spindle# rpm | % T | Solution Clarity | Polymer Solubility |
| 98 | 3.48 | 48.4 | #1, 60 | 96.6 | Clear | Control |
| 99 | 3.54 | 10.3 | #1, 60 | 96.2 | Clear | Control |
| 100 | 3.75 | 25.4 | #1, 60 | 96.9 | Clear | Soluble |
| 101 | 3.57 | 122.6 | #1, 30 | 95.5 | Clear | Soluble |
| 102 | 3.50 | 87.7 | #1, 60 | 96.5 | Clear | Soluble |
| 103 | 3.53 | 32.7 | #1, 60 | 95.4 | Clear | Soluble |
| 104 | 3.48 | 69.2 | #1, 60 | 96.3 | Clear | Soluble |
| 105 | 3.53 | 30.3 | #1, 60 | 96.4 | Clear | Soluble |

Process:
1. Weigh 80 g commercial product into 4 oz. wide mouth glass jars.
2. Add 20 g of a 1% polymer solution.
3. Cap jars and tape lid with electrical tape. Shake by hand to initially mix polymer.
4. Place and secure jars on tumbler. Use tape across jars and around jars on ends to prevent from tumbling over edge.
5. Tumble jars for 1.5 hours. After 1.5 hours, remove jars and temper in 25 C. bath overnight.
6. After overnight, remove jars from bath. Observe and record solution clarity and polymer solubility. Take pH and viscosity, measure % T at 600 nm. (24 hours sample) Store samples at ambient for 2 weeks and repeat temper in bath, observations, pH, viscosity, and % T.

What is claimed:

1. A conditioning composition comprising
(a) an aqueous based functional system selected from the group consisting of personal care products and household care products and
(b) a nonionic hydrophobically modified cellulose ether (HMCE) having a weight average molecular weight (Mw) with a lower limit of 400,000 and an upper limit of 2,000,000 and a hydrophobic substitution lower limit of 0.6 wt % and an upper limit amount which renders said cellulose ether insoluble in a 5 wt % solution of surfactant and less than 0.05% by weight soluble in water and wherein the cellulose ether provides conditioning benefit to a functional system substrate and
(c) at least one functional system active ingredient.

2. The composition of claim 1, wherein the (HMCE) forms an aqueous gel that is deposited upon the substrate upon dilution with water.

3. The composition of claim 1, wherein the upper limit of the Mw is 1,500,000.

4. The composition of claim 1, wherein the upper limit of the Mw is 1,000,000.

5. The composition of claim 1, wherein the lower limit of the Mw is 500,000.

6. The composition of claim 1, wherein the lower limit of the lower limit of the Mw is 600,000.

7. The composition of claim 1, wherein the lower limit amount of the hydrophobic substitution is 0.7 wt %.

8. The composition of claim 1, wherein the lower limit amount of the hydrophobic substitution is 0.8 wt %.

9. The composition of claim 1, wherein the hydrophobic moiety is selected from the group consisting of alkyl, aryl, alkyl aryl, and aryl alkyl.

10. The composition of claim 1, wherein the hydrophobic moiety is an alkyl having an upper limit amount of 30 carbons.

11. The composition of claim 1, wherein the hydrophobic moiety is an alkyl having an upper limit amount of 24 carbons.

12. The composition of claim 1, wherein the hydrophobic moiety is an alkyl having an upper limit amount of 18 carbons

13. The composition of claim 1, wherein the hydrophobic moiety is an alkyl having a lower limit amount of 3 carbons.

14. The composition of claim 1, wherein the hydrophobic moiety is an alkyl having a lower limit amount of 6 carbons.

15. The composition of claim 1, wherein the hydrophobic moiety is an alkyl having a lower limit amount of 8 carbons.

16. The composition of claim 1, wherein the hydrophobic moiety is an aryl, alkyl aryl, or aryl alkyl having a lower limit amount of 7 carbons.

17. The composition of claim 1, wherein the hydrophobic moiety is an aryl, alkyl aryl, or aryl alkyl having an upper limit amount of 30 carbons.

18. The composition of claim 1, wherein the hydrophobic moiety is cetyl.

19. The composition of claim 1, wherein the hydrophobic moiety is octyl.

20. The composition of claim 1, wherein the hydrophobic moiety is methylphenylglycidyl.

21. The composition of claim 1, wherein the hydrophobic moiety is butyl.

22. The composition of claim 1, wherein the hydrophobic moiety is 3-alkoxy-2-hydroxypropyl.

23. The composition of claim 22, wherein the alkoxy moiety has 3 to 30 carbons.

24. The composition of claim 1, wherein the HMCE has a backbone selected from the group consisting of hydroxyethylcellulose (HEC), hydroxypropylcellulose (HPC), ethyl hydroxyethylcellulose (EHEC), methyl hydroxyethylcellulose (MHEC), hydroxypropylmethylcellulose (HPMC), hydroxypropylhydroxyethylcellulose (HPHEC), ethyl hydroxypropylcellulose (EHPC), and methylcellulose (MC).

25. The composition of claim 1, wherein the hydrophobic moiety is attached to the backbone via an ether, ester, or urethane linkage.

26. The composition of claim 1, wherein the functional system substrate is selected from the group consisting of skin, hair, teeth, mucous membranes, textiles, and hard surfaces.

27. The composition of claim 26, wherein the hard surfaces are selected from the group consisting of metals, marbles, ceramics, granite, wood, hard plastics, and wall boards.

28. The composition of claim 1, wherein a surfactant is also present.

29. The composition of claim 28, wherein the surfactant is selected from anionic, nonionic, zwitterionic, or amphoteric surfactants.

30. The composition of claim 28, wherein the surfactant is present in an upper limit amount of 50 wt %.

31. The composition of claim 28, wherein the surfactant is present in a lower limit amount of 0.01 wt %.

32. The composition of claim 1, wherein a solvent is present and is selected from the group consisting of water-lower alkanols mixtures, polyhydric alcohols having 3 to 6 carbons and 2 to 6 hydroxyl groups.

33. The composition of claim 1, wherein the functional system is a personal care product that is selected from the group consisting of hair care, skin care, sun care, nail care, and oral care products.

34. The composition of claim 33, wherein the active personal care ingredient is selected from the group consisting of perfumes, skin coolants, emollients, moisturizer, deodorants, antiperspirants actives, moisturizing agents, cleansing agents, sunscreen actives, hair treatment agents, oral care agents, denture adhesive agents, shaving actives, beauty aids, and nail care active.

35. The composition of claim 33, wherein the personal care product is a hair care product that further comprises a conditioning agent selected from the group consisting of silicone materials, hydrocarbon oils, panthenol and derivatives thereof, pantothenic acid and derivatives thereof, and mixtures thereof.

36. The composition of claim 33, wherein the personal care composition is selected from the group consisting of body wash, shower gels, liquid soaps, bar soaps, skin lotions, skin creams, after shower lotions, after cleansing lotions, shave products, after shave products, deodorizing products, antiperspirant products, skin cleansing wipes, skin cooling wipes, skin conditioning wipes, skin drug delivery products, insect repellent products, sun care products, skin tanning products, skin coloring products, skin make-up products, eye care products, lipstick products, shampoos, conditioners, conditioning shampoo, hair styling products, hair coloring products, hair growth products, hair depilatory product, denture adhesive product, dental care products and mouth care products.

37. The composition of claim 33, wherein the personal care composition is an oil-in-water or water-in-oil emulsion or solution or slurry or dispersion or suspension.

38. The composition of claim 33, wherein the personal care product is a skin care product that further comprises a conditioning agent selected from the group of consisting of silicone materials, hydrocarbon oils, panthenol and derivatives thereof, pantothenic acid and derivatives thereof, and mixtures thereof.

39. The composition of claim 38, wherein the skin care product further comprises an emollient agent selected from the group consisting of polyhydric alcohols and hydrocarbons.

40. The composition of claim 33, wherein the composition further comprises at least one additional ingredient selected from the group consisting of hair-colorant, skin-colorant, skin-tanning agent, preservative, antioxidant, alpha or beta hydroxy acid, activity enhancer, emulsifier, functional polymer, viscosifying agent, alcohol, fat or fatty compound, antimicrobial compound, zinc pyrithione, silicone material, anti-dandruff, hydrocarbon polymer, emollient, oil, surfactant, flavor, fragrance, medicaments, rejunvenating agents, suspending agents, stabilizing biocides, and mixture thereof.

41. The composition of claim 1, wherein the functional system is household care composition that is selected from the group consisting of laundry detergent, dish washing products, heavy duty cleaning products, machinery lubricating products, disinfecting products, and fabric enhancing products, fabric softener, fabric abrasion reducing products, toilet cleaning products floor cleaning products, auto polishing products, auto cleaning products, shoe polish, shoe restoration products, paint remover products, household fragrance products and wall coloring products (PAINT), wall paper adhesives products.

42. The composition of claim 41, wherein the active household ingredient is selected from the group consisting of insect repellent agent, pet deodorizer agent, pet shampoo active, industrial grade bar and liquid soap active, dishwashing soap active, all purpose cleaner, disinfecting agent, grass and plant feeding agents, water treatment agent, rug and upholstery cleaning active, laundry softener active, laundry detergent active, toilet bowl cleaning agent, fabric sizing agent, fabric coloring agent, dust collection agent, antiredeposition agent, textile cleaning agent, softening, antistatic, and lubricating agent.

43. The composition of claim 41, wherein the composition further comprises at least one additional ingredient selected from the group consisting of colorant, preservative, antioxidant, bleaching agent, activity enhancer, emulsifier, functional polymer, viscosifying agent, alcohol, fat or fatty compound, oil, surfactant, fragrance, suspending agent, silicone material, and mixtures thereof.

44. The composition of claim 41, wherein the household composition is an oil-in-water or water-in-oil emulsion or solution or slurry or dispersion or suspension.

45. A process of conditioning an aqueous based functional system selected from the group consisting of personal care and household care products comprising adding and mixing a sufficient amount of a hydrophobically modified cellulose ether that is compatible with the aqueous based functional system to thicken the functional system wherein the hydrophobically modified cellulose ether is a nonionic hydrophobically modified cellulose ether (HMCE) polymer having a weight average molecular weight (Mw) with a lower limit of 400,000 and an upper limit of 2,000,000 and a hydrophobic substitution lower limit of 0.6 wt % and an upper limit amount which renders said cellulose ether insoluble in a 5 wt % solution of surfactant and less than 0.05% by weight soluble in water and wherein the cellulose ether provides conditioning benefit to a functional system substrate, and the resulting functional system has comparable or better conditioning properties as compared to when using similar thickening agents outside the scope of the present composition.

46. The process of claim 45, wherein the functional system has at least one active ingredient.

47. The process of claim 45, wherein the HMCE polymer forms an aqueous gel that is deposited upon the substrate upon dilution with water.

48. The process of claim 47 wherein the polymer concentration where the gel forms upon dilution with water has a lower limit of 0.05 wt %.

49. The process of claim 47 wherein the dilution with water has a lower limit of 0.25 wt %.

50. The process of claim 47 wherein the dilution with water has a lower limit of 1.0 wt %.

51. The process of claim 47 wherein the dilution with water has an upper limit of 5.0 wt %.

52. The process of claim 45, wherein the upper limit of the Mw is 1,500,000.

53. The process of claim 45, wherein the upper limit of the Mw is 1,000,000.

54. The process of claim 45, wherein the lower limit amount of the hydrophobic substitution is 0.7 wt %.

55. The process of claim 45, wherein the lower limit amount of the hydrophobic substitution is 0.8 wt %.

56. The process of claim 45, wherein the hydrophobic moiety is selected from the group consisting of alkyl, aryl, alkyl aryl, and aryl alkyl.

57. The process of claim 56, wherein the hydrophobic moiety is an alkyl having an upper limit amount of 30 carbons.

58. The composition of claim 56, wherein the hydrophobic moiety is an alkyl having an upper limit amount of 24 carbons.

59. The composition of claim 56, wherein the hydrophobic moiety is an alkyl having an upper limit amount of 18 carbons.

60. The composition of claim 56, wherein the hydrophobic moiety is an alkyl having a lower limit amount of 3 carbons.

61. The composition of claim 56, wherein the hydrophobic moiety is an alkyl having a lower limit amount of 6 carbons.

62. The composition of claim 56, wherein the hydrophobic moiety is an alkyl having a lower limit amount of 8 carbons.

63. The composition of claim 56, wherein the hydrophobic moiety is an aryl, alkyl aryl, or aryl alkyl having a lower limit amount of 7 carbons.

64. The composition of claim 56, wherein the hydrophobic moiety is an aryl, alkyl aryl, or aryl alkyl having an upper limit amount of 30 carbons.
